(19) United States
(12) Patent Application Publication
(10) Pub. No.: US 2021/0289820 A1
(43) Pub. Date: Sep. 23, 2021

(54) PRODUCTION OF ALCOHOL-FREE FERMENTED VEGETABLE JUICE WITH PICHIA KLUYVERI YEAST

(71) Applicant: Chr. Hansen A/S, Hoersholm (DK)

(72) Inventors: Sofie SAERENS, Hoersholm (DK); Jan Hendrik SWIEGERS, Hoersholm (DK)

(73) Assignee: Chr. Hansen A/S, Hoersholm (DK)

(21) Appl. No.: 17/266,050

(22) PCT Filed: Jul. 23, 2019
(86) PCT No.: PCT/EP2019/069780

§ 371 Date: Feb. 4, 2021

(30) Foreign Application Priority Data
Aug. 13, 2018 (EP) 18188644.1

(51) Int. Cl. A23L 2/84; A23L 2/02; A23L 2/38; C12N 1/16

(52) U.S. Cl. CPC A23L 2/84; A23L 2/02; C12R 2001/84; C12N 1/165; A23V 2002/00; A23L 2/382

(57) ABSTRACT

The invention relates to methods of preparing vegetable juice comprising fermenting a vegetable substrate with a Pichia kluyveri. Fermented vegetable juice obtained according the method has better flavor profiles, in particular reduced earthy flavors. The invention also provides Pichia kluyveri strains useful for fermenting vegetable substrates, which can be further processed to obtain beverages or other consumer products.

### FIELD OF THE INVENTION

The present invention relates to the preparations of low or non-alcoholic beverages of plant origins, and more particularly preparations of vegetable juices. The present invention also relates to the use of microorganisms to prepare such beverages.

### BACKGROUND OF THE INVENTION

Vegetables are a valuable dietary source of important nutrients required by the human body. Vegetable juice-based diets have become more and more popular. Vegetable juice is considered as a healthy alternative to fruit juice since it contains less sugar. The problem with vegetable juice, however, is that the flavor profile is not always appealing. The flavor of fresh vegetable juices can be earthy (such as in beetroot, carrot and cabbage juice) or sulfuric (such as in leek and onion juice), which is the main reason why vegetable juices are not nearly as palatable as fruit juices. It is a constant challenge to produce vegetable juice acceptable to a broad range of consumers involves balancing the flavor, aroma, appearance and satisfactory mouthfeel.

In addition, when vegetable juices are heat-treated for long term storage by pasteurization, more off-flavors are produced or enhanced during this process. Some of the off-flavors are described as cooked vegetables, which are not appealing for the consumers. To address this issue, the juice industry very often mixes vegetable juice into fruit juice.

Fermentation processes have been used to improve the taste of beverages. Corona et al. (Corona, Onofrio, et al. “Characterization of kefir-like beverages produced from vegetable juices.” LWT-Food Science and Technology 66 (2016): 572-581) describe fermenting vegetable and fruit juices with a kefir culture which contains both lactic acid bacteria and Saccharomyces cerevisiae. The lactic acid bacteria consist of Leuconostoc mesenteroides, Lactococcus lactis, Lactobacillus kefiri, and Lactobacillus fermentum. The fermented vegetable and fruit juices were analyzed by a sensory panel of 15 untrained panelists. The only juice that had a desirable flavor profile was the carrot kefir-like beverage. However, the carrot kefir-like beverage has an alcohol content of more than 3%, which is undesirable for a healthy beverage.

CN108244425 discloses a functional beverage prepared by fermenting beetroot (Beta vulgaris) juice with lactic acid bacteria and blending with tomato juice. Lactobacillus plantarum, Lactobacillus rhamnosus and Lactobacillus fermentum are used to ferment beetroot juice. Further ingredients including lemon extract, peppermint extract, fennel extract, honey and isomaltooligosaccharide are added to give aroma and a refreshing taste.

WO2012066176 describes the production of alcoholic fermented orange juice by use of the Pichia kluyveri strain deposited as CECT 13055. The strain is able to ferment the reducing sugars present in the orange juice, leading to a final product containing non-reducing sugars, such as sucrose, and thereby providing a product with pleasant tastes. Additionally, the final product typically has an alcoholic content around 2.5%.

WO2011134952 discloses inoculating Pichia kluyveri into grape juices. The yeast is added in frozen form is thawed to liquid form prior to being added to the medium. It has been found that frozen yeast can be used for direct inoculation without a significant loss in cell count and consequent activity. The reference does not describe any improvement of tastes of beverages.

US20170258113 discloses a method for preparing red bayberry fermented juice using Pichia kluyveri XT110. As disclosed, the Pichia kluyveri XT110 is able to ferment at a pH of between 2.5 to 3.0, which is important for keeping anthocyanin stable and therefore does not lead to serious color fading in the final product. The red bayberry fermented juice prepared has bright color, high content of anthocyanin, rich fruity taste and balanced flavor, and low content of alcohol (<0.5%).

WO2015117978 discloses using the two Pichia kluyveri strains PK-KR1 and PK-KR2 to prepare cider from apple and/or pear juice. The product was found to contain increased amounts of hexyl acetate and other desirable flavor compounds, particularly hexyl acetate and isobutyl acetate. The two Pichia kluyveri strains PK-KR1 and PK-KR2 were originally disclosed in WO2009110807, where it was described that they can be used to increased thiol levels (3MH and 3MHA) in wine fermentation process. 3MH and 3MHA have distinctive grapefruit- and passionfruit-like aromas and flavors. PK-KR1 and PK-KR2 were also used with different hop varieties to brew beer (WO2013030398) as well as low-alcohol or alcohol-free beer (WO2014135673).

However, no solution exists to enhance the flavor of vegetable juices. There is a need for new methods for preparing vegetable juices with desirable flavor profiles such as reduced unwanted off-flavors.

### SUMMARY OF THE INVENTION

The present invention provides a novel method for preparing vegetable juices with enhanced flavors. It has been discovered that Pichia kluyveri can be advantageously used to ferment vegetable juice to obtain a product with an improved flavor profile and therefore palatability.

As mentioned earlier, in contrast to fruit juice, the flavor profile of vegetable juice is not always appealing. The present invention therefore provides a different approach to improve its flavors without needing to needing mechanical techniques or mixing fruit juices.

In more details, it has been found that Pichia kluyveri can be used to produce increased levels of flavor compounds including isoamyl acetate, isobutyl acetate, β-phenylethanol, α-terpineol, c-whisky lactone and vanillin.

According to a sensorial evaluation, it is clearly shown that the earthy off-flavors are much less perceived in the fermented vegetable juices with Pichia kluyveri. The fermented juices also taste more fruity and spicy and are overall preferred compared to unfermented juices.

The present invention provides in a first aspect the use of Pichia kluyveri to prepare beverages comprising root vegetable material. It has been found that Pichia kluyveri can be advantageously used to treat vegetable juice, especially vegetable juice having undesired earthy flavor. Earthy flavor is common for vegetable juice prepared from plant material that was grown within or in contact with the soil. It is therefore desirable to enhance the flavor of vegetable juice by reducing the earthy taste. The present invention therefore provides another strategy for improving the flavor profile different from current methods used in the industry, which involves the use of mechanical techniques or mixing in fruit juices.

Preferred Pichia kluyveri include Pichia kluyveri strain 1, Pichia kluyveri strain 2 and Pichia kluyveri strain 3 as well as mutants thereof.

In another aspect, the invention provides the use of Pichia kluyveri to reduce the earthy flavor of plant material. The use involves fermenting plant material, especially root vegetables.

In a further aspect, the present invention provides methods of treating vegetable juice to reduce its earthy flavor and vegetable juice obtained therefrom. In one embodiment, the method comprises the steps of (a) providing a substrate comprising root vegetable material, (b) adding at least one Pichia kluyveri strain to the substrate, (c) fermenting the substrate, and (d) obtaining fermented vegetable juice. It should be understood that the substrate may comprise more than one type of root vegetables.

In other aspects, the present invention also provides vegetable juice obtained by the methods described in the application, as well as vegetable juice comprising one or more Pichia kluyveri, such as the Pichia kluyveri strains described herein.

The present invention additionally provides a new strain Pichia kluyveri as deposited 2014-03-05 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 28484.

### DEFINITIONS

As used herein, the term “vegetable” refers to an edible portion of a plant consumed by humans. The edible portion of a plant may be a root, such as rutabaga, beet, carrot, and sweet potato; a tuber or storage stem, such as potato and taro; the stem, as in asparagus and kohlrabi; a bud, such as Brussels sprouts; a bulb, such as onion and garlic; a petiole or leafstalk, such as celery and rhubarb; a leaf, such as cabbage, lettuce, parsley and spinach; an immature flower, such as cauliflower, broccoli and artichoke.

As used herein, the term “root vegetable” is edible portion of a plant vegetable which is in contact with the soil. Root vegetables are generally storage organs, enlarged to store energy in the form of carbohydrates. It should be noted that “root vegetables” as used herein is not limited to botanically defined root, but also includes both true roots (such as taproots and tuberous roots) and stems (such as bulbs, corms, rhizomes, and tubers).

In general, an “off-flavor” refers to the presence of objectionable tastes and/or odors in food. “Earthy” is a flavor which resembles the earth. Since this flavor is undesirable in food products, herein it is also referred to as “earthy off-flavors.” The odor of the earth has been attributed largely to the presence of geosmin (Gerber, N. N., and H. A. Lechevalier. “Geosmin, an earthy-smelling substance isolated from actinomycetes.” Applied microbiology 13.6 (1965): 935-938). Geosmin has been characterized in the soil (Buttery, Ron G., and John A. Garibaldi. “Geosmin and methylisoborneol in garden soil.” Journal of agricultural and food chemistry 24.6 (1976): 1246-1247) and is shown to be responsible for the earthy odor in root vegetable like beet (Acree, T. E., et al. “Geosmin, the earthy component of table beet odor.” Journal of Agricultural and Food Chemistry 24.2 (1976): 430-431).

Geosmin is however difficult to measure due to specific equipment required. However, the earthy flavor caused by geosmin can be reliably assessed using sensory analysis known in the art. Thus, “reducing earthy flavor” as used herein refers to a decrease the extent of the perception of the earthy flavor. This can be carried out by the comparing the earthy flavor before and after treatment with Pichia kluyveri. This can be evaluated by sensory evaluation methods known in the art, such as described in Meilgaard et al (Meilgaard, Morten C., B. Thomas Carr, and Gail Vance Civille. Sensory evaluation techniques. CRC press, 1999) or as described in the present application.

The earthy flavor may be measured with sensory analysis as described in the example.

The term “substrate” refers to a material that is fermentable by Pichia kluyveri.

The term “alcohol-free” herein refers to an alcohol content of less than 0.5% v/v.

The term “enhancing the flavor” of a product means to improve the flavor profile of a product so that it becomes more palatable. This can be determined for example by sensory assessment known to a skilled person in the art.

The term “fermentation” refers generally to any activity or process involving enzymatic decomposition (digestion) of organic materials by microorganisms. The term “fermentation” encompasses both anaerobic and aerobic processes, as well as processes involving a combination or succession of one or more anaerobic and/or aerobic stages.

The term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties as the mother strain. In the present context a mutant of the invention is preferable a mutant with same or improved properties with respect to reduction of earthy flavors. Such a mutant is a part of the present invention. A mutant may be a strain obtained by subjecting a strain of the invention to any conventionally used mutagenization treatment, including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 1000, no more than 100, no more than 20, no more than 10, or no more than 5, treatments are carried out. In a presently preferred mutant, less than 5%, or less than 1% or even less than 0.1% of the nucleotides in the bacterial genome have been changed (such as by replacement, insertion, deletion or a combination thereof) compared to the mother strain.

### BRIEF DESCRIPTION OF THE FIGURES

FIG. 1

Sensory analysis of beetroot juice prepared by fermentation with Pichia kluyveri strain 1 and Pichia kluyveri strain 2.

### DETAILED DESCRIPTION OF THE INVENTION

The present invention is in the technical field of alcohol-free beverages. It should be understood that such beverages have an ethanol concentration of less than 0.5% (v/v).

Driven by health-conscious consumers who are seeking to limit their alcohol consumption, the non-alcohol and low-alcohol sector is rapidly growing. In particular, vegetable juice is becoming increasing popular among health-oriented consumers. However, the flavor profile of vegetable juice is not always appealing. Thus, there is a constant need for providing products with improved flavor profiles for the market.

In a first aspect, the present invention provides a method of obtaining fermented vegetable juice, comprising adding at least one Pichia kluyveri strain to a substrate comprising root vegetable, and fermenting the substrate to obtain fermented vegetable juice. The present invention is based on the finding that Pichia kluyveri has advantageous properties useful in the treatment of vegetable juices, especially in the reduction of earthy off-flavors as well as enhancing the fruity and flowery flavors.

In one preferred embodiment, the method comprises:
a) providing a substrate comprising root vegetable,
b) adding at least one Pichia kluyveri strain to the substrate, and
c) fermenting the substrate comprising the Pichia kluyveri strain.

In another preferred embodiment, the method comprises:
a) providing a substrate comprising root vegetable,
b) adjusting the pH of the substrate,
c) adding at least one Pichia kluyveri strain to the substrate, and
d) fermenting the substrate comprising the Pichia kluyveri strain.

In a second aspect, the present invention also provides fermented vegetable juice obtained by the methods described herein.

The fermented vegetable juice is alcohol-free. As an example, the alcohol content (v/v) of the fermented vegetable juice is less than 0.5%, such as less than 0.4%, such as less than 0.3%, such as less than 0.2%, such as less than 0.1%, such as less than 0.09%, such as less than 0.08%.

It is contemplated that Pichia kluyveri is particularly useful for treating all vegetable which has earthy flavor. This includes especially root vegetables.

The earthy flavor, a characteristic trait of many vegetable juices, is caused by the aromatic compound geosmin. Geosmin (trans-1,10-dimethyl-trans-9-decalol) is a compound with very distinct earthy, musty, beetroot, even turnip flavor and has an extremely low sensory threshold of down to 10 ppm. It is a metabolite of various actinomycetes and fungi. It has been found to be responsible for the earthy taint in different foods, including fish and wine. Some bacteria such as Bacillus subtilis and Pseudomonas species have been reported to biodegrade geosmin. However, it has never been reported that fermentation with Pichia kluyveri can lead to a reduced perception of the off-flavor from geosmin.

To prepare a vegetable juice in accordance with the present invention, a substrate comprising root vegetable is provided.

The root vegetable can be derived from one or more types of vegetables. For example, the substrate can comprise material from one specific root vegetable or different types of root vegetables. In addition, the substrate can also additionally contain other plant material. For example, the substrate may comprise a mixture of type of root vegetables and fruits. In another embodiment, the substrate consists essentially of root vegetable.

The substrate according to the present invention comprises root vegetable. For example, the root vegetable is any one of the known species of root vegetables. Suitable root vegetables include, but are not limited to true roots, e.g., tuberous roots and taproots, and non-roots, e.g., tubers, rhizomes, corms and bulbs.

A true root vegetable can be a taproot and/or a tuberous root. Examples of taproots include, but are not limited to, arracachia (Arracacia xanthorrhiza), bush carrot (Abelmoschus moschatus), beet and mangelwurzel (Beta vulgaris), rutabaga and turnip (Brassica spp.), black cumin (Bunium persicum), burdock (Arctium), carrot (Daucus carota subsp. sativus), celeriac (Apium graveolens rapaceum), daikon (Raphanus sativus var. longipinnatus), dandelion (Taraxacum spp.), maca (Lepidium meyenii), yam daisy (Microseris scapigera), jicama and ahipa (Pachyrhizus spp.), parsnip (Pastinaca sativa), parsley root (Petroselinum spp.), radish (Raphanus sativus), black salsify (Scorzonera hispanica), skirret (Sium sisarum), salsify (Tragopogon spp.), and bush potato (Vigna lanceolata).

Examples of tuberous roots include, but are not limited to yellow lily yam (Amorphophallus galbra), pignut or earthnut (Conopodium majus), nagaimo, Chinese yam, Korean yam (Dioscorea opposita), native ginger (Hornstedtia scottiana), sweet potatoe (Ipomoea batatas), desert yam (Ipomoea costata), cassava or yuca or manioc (Manihot esculenta), mauka or chago (Mirabilis extensa), breadroot, tipsin, or prairie turnip (Psoralea esculenta), and yacon (Smallanthus sonchifolius).

An example of a root vegetable comprising a root-like stem is a Florida arrowroot (Zamia pumila).

A non-root comprises a tuber, a rhizome, a corm and/or a bulb. Examples of corm include, but are not limited to, konjac (Amorphophallus konjac), taro (Colocasia esculenta), Chinese water chestnut (Eleocharis dulcis), enset (Ensete spp.), water lilly (Nelumbo nucifera), arrowhead or wapatoo (Sagittaria spp.), yautia or malanga (Xanthosoma spp.).

Examples of rhizomes include, but are not limited to, turmeric (Curcuma longa), ginseng (Panax ginseng), rengarenga and vanilla lily (Arthropodium spp.), canna (Canna spp.), ti (Cordyline fruticosa), arrowroot (Maranta arundinacea), lotus root (Nelumbo nucifera), cattail or bulrush (Typha spp.), ginger, galangal (Zingiber officinale).

Examples of tubers include, but are not limited to, hog potato or groundnut (Apios americana), tigernut or chufa (Cyperus esculentus); yams, ube (Dioscorea spp.), Jerusalem artichoke or sunchoke (Helianthus tuberosus), daylily (Hemerocallis spp.), earthnut pea (Lathyrus tuberosus), New Zealand yam and oca (Oxalis tuberosa), kembili, dazo (Plectranthus edulis and P. esculentus), potato (Solanum tuberosum), Chinese artichoke or crosne (Stachys affinis), mashua or anu (Tropaeolum tuberosum), and ulluco (Ullucus tuberosus).

Examples of bulbs include, but are not limited to, garlic, onion, shallot, leek, etc. (Allium spp.), quamash (Camassia quamash), bush onion (Cyperus bulbosus), katakuri (Erythronium spp.), fennel (Foeniculum vulgare) and lilies (Lilium spp.),

Root vegetables can vary in their nutritional content, e.g., carbohydrate composition. For example, tubers can contain more starch than a true root vegetable, which can contain more simple sugars. Both true roots and non-roots are sources of other vitamins, minerals and other nutrients, such as fiber, potassium, vitamin A, calcium, vitamin B6, vitamin C, biotin, vitamin B1, vitamin B2, vitamin B3, iron, copper and magnesium.

Suitable root vegetable used for preparing the vegetable juice can be a monocotyledon, a dicotyledon, or a combination thereof. Examples of a monocotyledon (“monocot”) include a yam, such as a White Yam, Yellow Yam, Kokoro, Japanese Mountain Yam, Purple yam, Jicama, Malanga, or combinations thereof.

Examples of a dicotyledon include a sweet potato, a Batas, Brazilian sweet potatoes, Dingess sweet potatoes, Japanese Purple sweet potatoes, Okinawan sweet potatoes, Mokuau sweet potatoes, Covington sweet potatoes, Beauregard sweet potatoes.

The substrate described herein may comprise, for example, one or more types of root vegetables. For example, the substrate may comprise carrot and beetroot. In some embodiments, the substrate comprises 2, 3, 4, 5, 6, 7, 8, 9, 10 or more different types of root vegetables.

Preferred examples of root vegetables include, but are not limited to, carrot, beetroot, parsnip, swede, turmeric, ginger, potato, sweet potato, turnip, rutabaga, yuca, kohlrabi, onion, garlic, celery root, horseradish, daikon, jicama, radish, Jerusalem artichoke, and yam.

The process of preparing substrate can be outlined in the following way: washing the vegetable, optionally milling the vegetable into pulp and extracting (preferably by pressing) the juice of the vegetable without the pulp. Depending on the type of the vegetable, it may be pasteurized or sterilized if desired. Additional ingredients may also be present in the substrate.

In one embodiment, the vegetable juice may be acidified. This can be done by, for example, adding an acidification agent such as citric acid, lactic acid, lemon juice or the like. Alternatively, this could also be done by fermentation with lactic acid bacteria. Acidification may be used to stabilize the juice to reduce growth of pathogen in the final product.

Thus, in another embodiment the present invention provides a method comprising:
a) providing a substrate comprising root vegetable,
b) adjusting the pH of the substrate,
c) adding at least one Pichia kluyveri strain to the substrate, and
d) fermenting the substrate comprising the Pichia kluyveri strain.

Preferably, the pH is adjusted to between 2.5 and 6.5, such as between 2.5 and 6 or between 3 and 5.5. Pichia kluyveri is able to ferment the juice at these pH ranges. The pH of the substrate can be adjusted according to the product desired by the consumers.

The method in accordance with the present invention comprises the step of adding Pichia kluyveri to the substrate. This may include adding one or more Pichia kluyveri strains, such as 1, 2, 3, 4, 5 or more Pichia kluyveri strains. As used herein, a Pichia kluyveri “strain” means a genetic variant of Pichia kluyveri, which is readily determinable by a skilled person in the art based on its genetic makeup.

Pichia kluyveri is added to the substrate in an amount which is sufficient to initiate and maintain fermentation. The skilled person will readily be able to determine suitable concentrations of inoculation of the Pichia kluyveri according to the description and examples.

In preferred embodiments, Pichia kluyveri is inoculated in a concentration of at least 1×10⁴ CFU/ml, such as at least 5×10⁴ CFU/ml, such as at least 1×10⁵ CFU/ml, such as at least 5×10⁵ CFU/ml, such as at least 1×10⁶ CFU/ml, such as at least 5×10⁶ CFU/ml.

In some embodiments, a blend of more than one more strains, such as 2, 3, 4, 5 or more of Pichia kluyveri strains can be used.

Furthermore, the Pichia kluyveri added may be in frozen, liquid or dried form, including e.g. freeze-dried form and spray/fluid bed dried form, or frozen or freeze-dried concentrates.

The method in accordance with the present invention comprises the step of fermenting the substrate comprising the added Pichia kluyveri. Fermentation of the substrate is preferably carried out by controlled fermentation in sterile settings. During the fermentation process, a skilled person in the art is able to choose to exercise several other options known to him, such as but not limited to, the addition of pectinase during milling/pressing, adjustment of pH, addition of sulphur dioxide as an antimicrobial agent, fining and filtration, in order to achieve the desired end-product.

Preferably, the fermentation of the substrate with Pichia kluyveri is carried out for at least 12 hours, such as for 18 hours, such as for 24 hours, such as for 36 hours, such as for 48 hours. It is preferable that the fermentation does not exceed 48 hours, because the alcoholic content may increase to a level which is undesired for the intended end-product. A skilled person in the art can readily select suitable fermentation time based on the desired properties of the products.

In some embodiments, the fermentation does not exceed 36 hours. In other embodiments, the fermentation is carried out between 12 and 48 hours.

Preferably, the fermentation condition using Pichia kluyveri is semi-anaerobic. Such fermentation may start aerobically and proceeds anaerobically after all oxygen is consumed.

In some embodiments of the present invention Pichia kluyveri is added to the substrate as a starter culture. The term “starter culture” refers to a composition comprising live microorganisms that are capable of initiating or effecting fermentation of organic material, optionally after being cultivated in a separate starter medium for obtaining a high-density culture. Accordingly, in an embodiment, a starter culture of the invention may be a high-density culture obtained by propagating a starter culture in a suitable medium.

A starter culture according to the present invention may also contain, in addition to microorganisms, buffering agents and growth stimulating nutrients or preservatives or other carriers.

In a preferred embodiment the first starter culture contains at least 10² colony forming units (CFU)/g of Pichia kluyveri, such as at least 10³ CFU/g, such as at least 10⁴ CFU/g, such as at least 10⁵ CFU/g, such as at least 10⁶ CFU/g, such as at least 10⁷ CFU/g, such as at least 10⁸ CFU/g, such as at least 10⁹ CFU/g, such as at least 10¹⁰ CFU/g, such as at least 10¹¹ CFU/g, such as at least 10¹² CFU/g, such as at least 10¹³ CFU/g.

The fermentation may be carried out at a temperature of between 4-35° C. In preferred embodiments the fermentation is carried out at a temperature of between 18 and 22° C., such as between 19-21° C., such as 20° C.

Fermentation can be terminated by any suitable methods known in the art, including cooling down, preferably to below 4° C., and/or pasteurization. Fermented vegetable juice is then obtained. Preferably, the fermentation liquid is separated from the solids using any suitable methods known in the art, for example by filtration or centrifugation.

Depending on the product, one may further include additional components in the fermented juice, such as thickeners, sugar, sweetener, dietary fibers, syrup, buffers, pH adjusting agents, salt, essential minerals, pectin, proteins, flavorants, colorants, vitamins, preservatives, antioxidants, etc.

It is also envisioned that the fermented liquid can be further processed, for example by concentration, dilution or mixing with other products.

The fermented juice is preferably filled in bottles and optionally pasteurized. In other embodiments the fermented juice is not pasteurized.

Acetate esters are important flavor compounds in beverages. For example, the best-known acetate esters are isoamyl acetate and ethyl acetate. Ethyl acetate is a fruity smelling liquid with a brandy note and is common ester in fruits. The inventors have found that the level of ethyl acetate in the vegetable juice with Pichia kluyveri is increased after the fermentation. In some embodiments, ethyl acetate level is at least 10 ppm, such as at least 12 ppm, at least 13 ppm, at least 14 ppm, at least 15 ppm, at least 16 ppm, at least 17 ppm, at least 18 ppm, at least 19 ppm, at least 20 ppm, at least 21 ppm, at least 22 ppm, at least 23 ppm, at least 24 ppm, at least 25 ppm, at least 26 ppm, at least 27 ppm, at least 28 ppm, at least 29 ppm, or at least 30 ppm.

Isoamyl acetate, also known as isopentyl acetate, is an organic compound that is the ester formed from isoamyl alcohol and acetic acid. Isoamyl acetate occurs naturally in the banana plant. It has a strong odor which is also described to resemble banana and pear smell. However, at high amount it generates an obvious acetone like off-odor which can be perceived as unpleasant.

The inventors have found that the level of isoamyl acetate in the vegetable juice with Pichia kluyveri is increased after the fermentation. In some preferred embodiments, the isoamyl acetate in the fermented vegetable juice prepared according to the present invention is at least 2 ppm, such as at least 2.1 ppm, at least 2.2 ppm, at least 2.3 ppm, at least 2.4 ppm, at least 2.5 ppm, at least 2.6 ppm, at least 2.7 ppm, at least 2.8 ppm, at least 2.9 ppm, at least 3.0 ppm, at least 3.1 ppm, at least 3.2 ppm, at least 3.3 ppm, at least 3.4 ppm, at least 3.5 ppm, at least 3.6 ppm, at least 3.7 ppm, at least 3.8 ppm, at least 3.9 ppm, or at least 4.0 ppm.

The weight ratio of isoamyl acetate to ethyl acetate a good indication of fruitiness. Like ethanol, ethyl acetate is the ester of ethanol and acetic that acts as a solvent. It contributes to the release of the flavor of isoamyl acetate.

The inventors have found that such weight ratio in the vegetable juice fermented with Pichia kluyveri is increased after the fermentation. In some preferred embodiments, the weight ratio of isoamyl acetate to ethyl acetate of the fermented juice prepared according to the present invention is at least 0.1, such as at least 0.11, at least 0.12, at least 0.13, at least 0.14, at least 0.15, at least 0.16, at least 0.17, at least 0.18, at least 0.19, or at least 0.2. Preferably, the weight ratio of isoamyl acetate to ethyl acetate of the fermented juice is at least 0.2, such as at least 0.21, at least 0.22, at least 0.23, at least 0.24, at least 0.25, at least 0.26, at least 0.27, at least 0.28, at least 0.29, or at least 0.30.

The inventors have found that such weight ratio of isoamyl acetate to ethanol in the vegetable juice fermented with Pichia kluyveri is increased after the fermentation. In some preferred embodiments, the weight ratio of isoamyl acetate to ethanol of the fermented juice prepared according to the present invention is at least 0.0002, such as at least 0.0003, at least 0.0004, at least 0.0005, at least 0.0006, at least 0.0007, at least 0.0008, at least 0.0009, at least 0.001, at least 0.0011, at least 0.0012 or more.

It has been found that vegetable juice fermented with Pichia kluyveri contains more flavor compounds, including β-phenylethanol and α-terpineol. β-phenylethanol has a rose-like flavor and works synergistically with other flavor compounds like esters to enhance the overall fruity impression. The inventors have found that β-phenylethanol in the vegetable juice fermented with Pichia kluyveri is increased after the fermentation. In some preferred embodiments, the β-phenylethanol in the fermented vegetable juice is as at least 0.2 ppm, such as at least 0.3 ppm, such as at least 0.4 ppm, such as at least 0.5 ppm, such as at least 0.6 ppm, such as at least 0.7 ppm.

α-terpineol has a pleasant odor similar to lilac. It is a terpene alcohol that is found in natural oils such as pine oil. It is also one of the most commercially important monoterpene alcohols for the flavor industry. The inventors have found that α-terpineol in the vegetable juice fermented with Pichia kluyveri is increased after the fermentation. In some preferred embodiments, the α-terpineol in the fermented vegetable juice is at least 5 ppb, such as at least 10 ppb, such as at least 20 ppb, such as at least 30 ppb, such as at least 40 ppb, such as at least 50 ppb, such as at least 60 ppb, or such as at least 70 ppb.

c-whisky lactone (cis-whisky lactone), also known as (3S,4S)-3-methyl-4-octanolide, is an important ingredient in the aroma of whiskey and other alcoholic beverages that have been aged in oak barrels. It is sometimes added to the liquor as a flavoring agent. The inventors have found that c-whisky lactone in the vegetable juice fermented with Pichia kluyveri is increased after the fermentation.

In some preferred embodiments, the c-whisky lactone in the fermented vegetable juice is at least 1 ppb, such as at least 2 ppb, such as at least 3 ppb, such as at least 4 ppb, such as at least 5 ppb, such as at least 6 ppb, such as at least 7 ppb.

Vanillin, a phenolic aldehyde, is the principal flavor and aroma compound in vanilla. With its sweet and creamy vanilla odor, vanillin is used more often than natural vanilla extract as a flavoring agent in foods, beverages, and pharmaceuticals. The inventors have been found that vanillin in the vegetable juice fermented with Pichia kluyveri is increased after the fermentation. In some preferred embodiments, the vanillin in the fermented vegetable juice is at least 2 ppb, such as at least 3 ppb, such as at least 4 ppb, such as at least 5 ppb, such as at least 6 ppb, such as at least 7 ppb.

In a further aspect the present invention provides fermented vegetable juice having improved flavor profiles. A vegetable juice prepared according to the present invention comprises root vegetable and Pichia kluyveri, which can be for example Pichia kluyveri strain 1, Pichia kluyveri strain 2, Pichia kluyveri strain 3, mutants thereof or any of the combinations thereof.

Pichia kluyveri strain 1 was deposited on 5 Mar. 2014 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 28484.

Pichia kluyveri strain 2 was deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02271.

Pichia kluyveri strain 3 was deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02272.

In a further aspect the present invention provides use of Pichia kluyveri to ferment root vegetables. In some embodiments, Pichia kluyveri strain 1, Pichia kluyveri strain 2, Pichia kluyveri strain 3, mutants thereof or any of the combinations thereof is used to ferment root vegetables to obtain vegetable juice as described in the present application.

The inventors have shown the Pichia kluyveri strains have a huge effect on the flavor profile of the final fermented juice product. Huge increases were surprisingly found for all desirable flavor compounds, such as esters and terpenes. As shown in the examples, the flavor analysis corresponds well with the sensory evaluation. The sensory evaluation demonstrated that vegetable juices fermented with Pichia kluyveri have much less earthy off-flavor and more fruity and spicy aroma compared to an unfermented control.

The present invention additionally provides a product which is Pichia kluyveri strain 1 in frozen, freeze-dried, or liquid form, including e.g. freeze-dried form and spray/fluid bed dried form, or frozen or freeze-dried concentrates. Preparation of the different forms are known in the art and for example described in WO2011/134952 (Chr. Hansen A/S). Pichia kluyveri may be grown in in a fermenter and concentrated. Additionally, cryoprotectants can be added to maintain the viability of the yeast under a low temperature. The product can be directly added to the substrate comprising root vegetable.

Furthermore, the present invention provides a composition for preparing fermented beverages, comprising Pichia kluyveri strain 1.

Items

The following items are preferred embodiments of the present invention:
1. A method for reducing the earthy off-flavor of vegetable juice, comprising the steps of
   a) providing a substrate comprising root vegetable,
   b) adding at least one Pichia kluyveri strain to the substrate,
   c) fermenting the substrate comprising the Pichia kluyveri strain for less than 48 hours,
   d) obtaining fermented vegetable juice.
2. The method of item 1, wherein the root vegetable is select from the group consisting of carrot, beetroot, parsnip, swede, turmeric, ginger, potato, sweet potato, turnip, rutabaga, yuca, kohlrabi, onion, garlic, celery root, horseradish, daikon, jicama, radish, Jerusalem artichoke, and yam.
3. The method of any of the preceding items, wherein the Pichia kluyveri is selected from the group consisting of
   a) Pichia kluyveri deposited 5 Mar. 2014 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 28484,
   b) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02271,
   c) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02272, and
   d) mutants of a), b) or c).
4. The method of any of the preceding items, wherein the weight ratio of isoamyl acetate to ethyl acetate at least 0.1, preferably at least 0.2,
5. The method of any of the preceding items, wherein step b) further comprises adding a lactic acid bacteria strain to the substrate.
6. The method of any of the preceding items, wherein the fermentation in step c) is carried out at a temperature between 4 and 35° C., more preferably between 18 and 22° C.
7. The method of any of the preceding items, wherein β-phenylethanol in the fermented vegetable juice is at least 0.4 ppm.
8. The method of any of the preceding items, wherein α-terpineol in the fermented vegetable juice is at least 50 ppb.
9. The method of any of the preceding items, wherein c-whisky lactone in the fermented vegetable juice is at least 4 ppb.
10. The method of any of the preceding items, wherein vanillin in the fermented vegetable juice is at least 4 ppb.
11. Fermented vegetable juice obtained by the methods according to any of the preceding items.
12. Fermented vegetable juice comprising root vegetable and Pichia kluyveri.
13. The fermented vegetable juice according to item 12, wherein the Pichia kluyveri is selected from the group consisting of:
   a) Pichia kluyveri deposited 5 Mar. 2014 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 28484,
   b) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02271,
   c) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02272, and
   d) mutants of a), b) or c).
14. The fermented vegetable juice according to any of the items 12-13, wherein the weight ratio of isoamyl acetate to ethyl acetate is at least 0.1, preferably at and/or wherein the weight ratio of isoamyl acetate to ethanol is at least 0.0002, preferably at least 0.0008.
15. The fermented vegetable juice according to any of the items 12-14, wherein β-phenylethanol in the fermented vegetable juice is at least 0.4 ppm.
16. The fermented vegetable juice according to any of the items 12-15, wherein α-terpineol in the fermented vegetable juice is at least 50 ppb.
17. The fermented vegetable juice according to any of the items 12-16, wherein c-whisky lactone in the fermented vegetable juice is at least 4 ppb.
18. The fermented vegetable juice according to any of the items 12-17, wherein c vanillin in the fermented vegetable juice is at least 4 ppb.
19. Use of Pichia kluyveri to ferment root vegetable.
20. The use of item 19, wherein the root vegetable is select from the group consisting of carrot, beetroot, parsnip, swede, turmeric, ginger, potato, sweet potato, turnip, rutabaga, yuca, kohlrabi, onion, garlic, celery root, horseradish, daikon, jicama, radish, Jerusalem artichoke, and yam.
21. The use of item 19 or 20, wherein the Pichia kluyveri is selected from the group consisting of:
   a) Pichia kluyveri deposited 5 Mar. 2014 at German Collection of Microorganisms and Cell Cultures (Deutsche Sammlung von Mikroorganismen und Zellkulturen GmbH; DSMZ), Inhoffenstr. 7B, D-38124 Braunschweig and given the accession No.: DSM 28484,
   b) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02271,
   c) Pichia kluyveri deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02272, and
   d) mutants of a), b) or c).
22. Pichia kluyveri as deposited 5 Mar. 2014 at German Collection of Microorganisms and Cell Cultures (Deutsche Sammlung von Mikroorganismen und Zellkulturen GmbH; DSMZ), Inhoffenstr. 7B, D-38124 Braunschweig and given the accession No.: DSM 28484 and mutants thereof.
23. A composition for fermentation comprising the Pichia kluyveri of item 22.
24. The composition according to item 23, wherein the composition is in frozen, freeze-dried, or liquid form.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit and Expert Solution

The applicant requests that a sample of the deposited microorganisms stated below may only be made available to an expert, until the date on which the patent is granted.

Pichia kluyveri strain 1 was deposited at DSMZ (Deutsche Sammlung von Mikroorganismen and Zellkulturen GmbH, Inhoffenstrasse 7B, D-38124 Braunschweig) on 5 Mar. 2014 by Chr. Hansen A/S, Denmark and was given the accession number DSM 28484. The deposit has been made under the conditions of the Budapest Treaty on the International Recognition of the Deposit of Microorganisms for the Purposes of Patent Procedure.

Pichia kluyveri strain 2 (PK-KR1) and Pichia kluyveri strain 3 (PK-KR2) were deposited on 24 Aug. 2006 at the National Measurement Institute, 541-65 Clarke Street, South Melbourne, Victoria 3205, Australia, by University of Auckland, School of Biological Sciences, Auckland 1142, New Zealand, and given the accession numbers V06/02271 and V06/02272, respectively, as described in WO 2009/110807.

### EXAMPLES

### Example 1

Carrot juice with a pH of 4.5 was provided as vegetable substrate. It was prepared by diluting carrot juice concentrate.

Lab-scale fermentations (200 ml) were carried out. Carrot juice was inoculated with two different Pichia kluyveri, namely, Pichia kluyveri strain 1 (DSM 28484) and strain 2 (deposited at the National Measurement Institute under the accession number V06/022711) at 10⁵ CFU/ml. The fermentations were placed in an incubator at 20° C. for 24 hours. Afterwards samples were taken.

Ethanol Analysis

Ethanol was measured with the Ethanol Enzymatic Bioanalysis kit of Boehringer Mannheim. Ethanol concentrations of the fermentation products after 24 h of fermentation with Pichia kluyveri strain 1 and strain 2. Table 1 shows the results of the ethanol concentrations in unfermented and fermented carrot juice.

TABLE 1
Ethanol concentration before and after fermentation

| | EtOH % | EtOH |
|---|---|---|
| Samples | (v/v) | (ppm) |
| Unfermented | 0.12 | 946.8 |
| Fermented with Pichia kluyveri strain 1 | 0.34 | 2682.6 |
| Fermented with Pichia kluyveri strain 2 | 0.30 | 2367 |

As shown, the ethanol concentrations in the final fermented carrot product were below 0.05% (v/v).

Flavor Analysis

Flavor analysis of all the fermentation products was carried out at the Lab for Flavor Analysis and Enology in Zaragoza, Spain. Table 2-6 shows the concentrations of different flavor compounds in the samples.

TABLE 2
Ethyl acetate concentrations before and
after fermentation

| | ethyl acetate |
|---|---|
| Samples | (ppm) |
| Unfermented | 0.00 |
| Fermented with Pichia kluyveri strain 1 | 10.30 |
| Fermented with Pichia kluyveri strain 2 | 25.06 |

TABLE 3
Acetate ester (isoamyl acetate and isobutyl acetate)
concentrations before and after fermentation

| | isoamyl | isobutyl |
|---|---|---|
| | acetate | acetate |
| Samples | (ppm) | (ppm) |
| Unfermented | 0.12 | 0.01 |
| Fermented with Pichia kluyveri strain 1 | 2.27 | 0.07 |
| Fermented with Pichia kluyveri strain 2 | 2.56 | 0.18 |

TABLE 4
Isoamyl acetate/ethyl acetate ratio before and
after fermentation

| | Ratio of isoamyl |
|---|---|
| | acetate (ppm) to |
| Samples | ethyl acetate (ppm) |
| Unfermented | 0.00 |
| Fermented with Pichia kluyveri | 0.22 |
| strain 1 | |
| Fermented with Pichia kluyveri | 0.10 |
| strain 2 | |

TABLE 5
Isoamyl acetate/ethanol ratio before and
after fermentation

| | Ratio of isoamyl |
|---|---|
| | acetate (ppm) to |
| Samples | ethanol (ppm) |
| Unfermented | 0.00013 |
| Fermented with Pichia kluyveri strain 1 | 0.00085 |
| Fermented with Pichia kluyveri strain 2 | 0.00108 |

TABLE 6
β-phenylethanol concentrations before and
after fermentation

| | β-phenylethanol |
|---|---|
| Samples | (ppm) |
| Unfermented | 0.10 |
| Fermented with Pichia kluyveri strain 1 | 0.86 |
| Fermented with Pichia kluyveri strain 2 | 0.44 |

TABLE 7
α-terpineol concentrations before and after fermentation

| | α-terpineol |
|---|---|
| Samples | (ppb) |
| Unfermented | 2.67 |
| Fermented with Pichia kluyveri strain 1 | 55.77 |
| Fermented with Pichia kluyveri strain 2 | 51.52 |

TABLE 8
aroma compounds (c-whiskylactone and vanillin)
concentrations before and after fermentation

| | c-whiskylactone | Vanillin |
|---|---|---|
| Samples | (ppb) | (ppb) |
| Unfermented | 0.00 | 1.24 |
| Fermented with Pichia kluyveri | 5.44 | 4.31 |
| strain 1 | | |
| Fermented with Pichia kluyveri | 4.91 | 4.17 |
| strain 2 | | |

Table 2-4 clearly show that fermentation of the carrot juice with Pichia kluyveri enhances the ester profile in the final fermented product. After only 24 h of fermentation, ethyl acetate, isoamyl and isobutyl acetate concentrations were increased at least two-fold in the fermented carrot juices.

Pichia kluyveri was shown to increase the ratio of isoamyl acetate (ppm) to ethyl acetate. Pichia kluyveri strain 1 produced less ethyl acetate than Pichia kluyveri strain 2, but similar amounts of isoamyl acetate. As a result, the ratio of isoamyl acetate/ethyl acetate is higher in Pichia kluyveri strain 1.

In addition, Pichia kluyveri was shown to increase the ratio of isoamyl acetate (ppm) to ethanol (ppm). Pichia kluyveri strain 1 showed a lower isoamyl acetate (ppm)/ethanol (ppm) compared to Pichia kluyveri strain 2, but similar amounts of isoamyl acetate.

Table 6-7 show the concentrations of two flavor compounds, β-phenylethanol and α-terpineol, respectively. Both compounds increased significantly in the fermented product and contributed to its flowery and spicy notes. Pichia kluyveri strain 1 produces higher amount of β-phenylethanol and α-terpineol than strain 2.

In addition to esters, alcohols and terpene, the inventors have also discovered some lactones and even vanillin were increased, imparting a more a sweeter flavor the fermented product. The concentrations of c-whiskylactone and vanillin are shown in Table 8.

### Example 2

Fresh, unacidified beetroot juice was provided as vegetable substrate. It was prepared by pressing beetroot. Lab-scale fermentations (400 ml) were carried out. The beetroot juice was inoculated with the same Pichia kluyveri strains, P. kluyveri strain 1 and P. kluyveri strain 2, as described in Example 1. The fermentations were placed in an incubator at 20° C. for 24 hours. Afterwards samples were taken.

Sensory Analysis

A sensory evaluation using the descriptive analysis technique according to Lawless et al (Lawless, H. T., and H. Heymann. “Sensory Evaluation of Food: Practices and Principals.” Food Science Texts Series. Chapman and Hall. New York (2010)) was conducted to assess how Pichia kluyveri contributes to the flavor profile.

After the fermentation, four apparent attributes of the fermented beetroot juices were determined. These attributes were: earthy, overall fruity, spicy and sweet. Nine panelists were chosen and a training session with these attributes was conducted. At the training session the panelists were trained in recognizing the attributes' aroma and taste. This was done by smelling raw beetroot for the earthy attribute, pepper for the spicy attribute, overall fruity by smelling tropical fruit juice and sweet by smelling carrot juice.

After the training session, the panelists were invited to a tasting session of the fermented juices. The different juices along with non-fermented beetroot juice were appointed with three random numbers and served to each judge. The juice was served at approximately 10° C. in wine glasses. The panelists scored the 4 flavors (aroma and taste combined) of the juices, by scaling on a 145 mm scale (0 representing not present, 145 as highly present) as well as for their subjective, overall liking/acceptance of the juice.

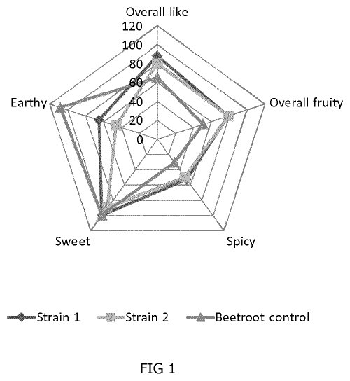

FIG. 1 shows the results of the sensorial evaluation. The figure shows how the different juices were evaluated on a scale from 0-145 for five different attributes: earthy, overall fruity, spicy, sweet and overall like.

FIG. 1 demonstrates that all the juices fermented by Pichia kluyveri were evaluated to be significantly less earthy compared to the unfermented beetroot juice (beetroot control in FIG. 1). Furthermore, the sweetness level of the fermented juices was very similar to the unfermented beetroot control. They were additionally perceived as more fruity and spicy compared to control.

The fermented beetroot juice by Pichia kluyveri is overall more preferred than the unfermented beetroot juice due to reduced earthy off-flavor and increased fruity flavor. Pichia kluyveri strain 1 was overall more preferred than Pichia kluyveri strain 2, even though the earthy off-flavor was a perceived to be slightly higher in the beetroot juice fermented with Pichia kluyveri strain 1 compared to strain 2.

The highest flavor differences were found for the esters and α-terpineol. A look closer to the acetate ester concentrations reveals that the ratio of isoamyl acetate/ethyl acetate, a good measure of fruitiness, is increased in both Pichia kluyveri strains. This corresponds well with the sensory evaluation which demonstrated that fermentation of vegetable juice resulted in higher scores for fruitiness and spiciness, and much lower scores for earthy off-flavors, compared to unfermented beetroot juice.

1. A method for reducing the earthy off-flavor of vegetable juice, comprising:
(a) adding at least one Pichia kluyveri strain to a substrate comprising a root vegetable, and
(b) fermenting the substrate comprising the Pichia kluyveri strain,
thereby obtaining fermented vegetable juice.

2. The method of claim 1, wherein the root vegetable is select from the group consisting of carrot, beetroot, parsnip, swede, turmeric, ginger, potato, sweet potato, turnip, rutabaga, yucca, kohlrabi, onion, garlic, celery root, horseradish, daikon, jicama, radish, Jerusalem artichoke, and yam.

3. The method of claim 1, wherein the at least one Pichia kluyveri strain comprises one or more selected from the group consisting of:
(a) the Pichia kluyveri strain deposited at the Deutsche Sammlung von Mikroorganismen and Zellkulturen GmbH (DSMZ), Inhoffenstr. 7B, D-38124 Braunschweig, under Accession No. DSM 28484;
(b) the Pichia kluyveri strain deposited at the National Measurement Institute, 541-65 Clarke Street, South Melbourne, Victoria 3205, Australia, under Accession No. V06/02271;
c) the Pichia kluyveri strain deposited at the National Measurement Institute, 541-65 Clarke Street, South Melbourne, Victoria 3205, Australia, under Accession No. V06/02272, and
d) mutants of any of (a), (b) or (c).

4. The method of claim 1, wherein the fermented vegetable juice comprises isoamyl acetate and ethyl acetate in a weight ratio of isoamyl acetate to ethyl acetate of at least 0.1.

5. The method of claim 1, adding a lactic acid bacteria strain to the substrate prior to the fermenting.

6. The method of claim 1, wherein the fermenting is carried out at a temperature between 4° C. and 35° C.

7. The method of claim 1, wherein the fermented vegetable juice comprises β-phenylethanol in an amount of at least 0.4 ppm.

8. The method of claim 1, wherein the fermented vegetable juice comprises α-terpineol in an amount of at least 50 ppb.

9. The method of claim 1, wherein the fermented vegetable juice comprises one or both of c-whisky lactone and vanillin in an amount of at least 4 ppb.

10. Fermented vegetable juice obtained by the method of claim 1, wherein the fermented vegetable juice comprises Pichia kluyveri.

11. Fermented vegetable juice comprising fermented root vegetable juice and Pichia kluyveri.

12. The fermented vegetable juice according to claim 11, wherein the Pichia kluyveri is selected from the group consisting of:
(a) the Pichia kluyveri strain deposited at the DSMZ under Accession No. DSM 28484;
(b) the Pichia kluyveri strain deposited at the National Measurement Institute, 541-65 Clarke Street, South Melboune, Victoria 3205, Australia, under Accession No. V06/02271;
(c) the Pichia kluyveri strain deposited at the National Measurement Institute, 541-65 Clarke Street, South Melbourne, Victoria 3205, Australia, under Accession No. V06/02272, and
(d) mutants of any of (a), (b) or (c).

13-14. (canceled)

15. The Pichia kluyveri strain deposited at the DSMZ under Accession No. DSM 28484 and mutants thereof.

16. The method of claim 1, wherein the substrate is a root vegetable juice, and wherein the fermented vegetable juice has a reduced earthy off-flavor as compared to the vegetable juice prior to the fermentation with Pichia kluyveri.

17. The method of claim 1, wherein the fermented vegetable juice comprises isoamyl acetate and ethyl acetate in a weight ratio of isoamyl acetate to ethyl acetate of at least 0.2.

18. The method of claim 1, wherein the fermented vegetable juice comprises isoamyl acetate and ethanol in a weight ratio of isoamyl acetate to ethanol of at least 0.0002.

19. The method of claim 1, wherein the fermented vegetable juice comprises isoamyl acetate and ethanol in a weight ratio of isoamyl acetate to ethanol of at least 0.0008.

20. The method of claim 1, wherein the fermenting is carried out at a temperature between 18° C. and 22° C.
